SEQUENCE LISTING (30 sequences)

SEQ ID NO: 1 - DNA, 18 bp, Staphylococcus I
cacatcagcgtcagttac

SEQ ID NO: 2 - DNA, 18 bp, Staphylococcus II
cacatcagcgtcagttgc

SEQ ID NO: 3 - DNA, 18 bp, Staphylococcus III
aagcttaagggttgcgct

SEQ ID NO: 4 - DNA, 18 bp, Peptostreptococcus I
gccttctaaatcacgcgg

SEQ ID NO: 5 - DNA, 18 bp, Peptostreptococcus II
agcccaagtcataaaggg

SEQ ID NO: 6 - DNA, 18 bp, Peptostreptococcus III
tacactctctcaagccgg

SEQ ID NO: 7 - DNA, 18 bp, Corynebacterium I
agcactcaagttatgccc

SEQ ID NO: 8 - DNA, 18 bp, Corynebacterium II
agtactcaagttatgccc

SEQ ID NO: 9 - DNA, 18 bp, Corynebacterium III
agcactcaagtaatgccc

SEQ ID NO: 10 - DNA, 18 bp, Corynebacterium IV
  misc_feature (14)..(14): n = a, c, g or t
agcactcaagtcangccc

SEQ ID NO: 11 - DNA, 18 bp, Corynebacterium V
agcactctagttatgccc

SEQ ID NO: 12 - DNA, 18 bp, Corynebacterium VI
ggccggctttcagcgatt

SEQ ID NO: 13 - DNA, 18 bp, Veillonella I
gcttccatcgctcttcgt

SEQ ID NO: 14 - DNA, 18 bp, Veillonella II
gttctgtccatcaatgtc

SEQ ID NO: 15 - DNA, 18 bp, Veillonella III
ttccgtctattaactccc

SEQ ID NO: 16 - DNA, 18 bp, Propionibacterium acnes
tcacgcttcgtcacaggc

SEQ ID NO: 17 - DNA, 18 bp, Propionibacterium acnes
caggctcgccactctctg

SEQ ID NO: 18 - DNA, 19 bp, Malassezia
tacggcgattccaaaaacc

SEQ ID NO: 19 - DNA, 18 bp, Corynebacterium VII
cacactaaaaatggctcc

SEQ ID NO: 20 - DNA, 18 bp, Corynebacterium VIII
tccacaccatggtcctat

SEQ ID NO: 21 - DNA, 18 bp, Corynebacterium IX
ccatccaaaatgcggtcc

SEQ ID NO: 22 - DNA, 18 bp, Corynebacterium X
ccatccaaaatgtggtcc

SEQ ID NO: 23 - DNA, 19 bp, Corynebacterium XI
caccatccaaaatgtggtc

SEQ ID NO: 24 - DNA, 19 bp, Corynebacterium XII
caccatccaaaatgcggtc

SEQ ID NO: 25 - DNA, 17 bp, Corynebacterium XIII
ctgcagtcccgcagtta

SEQ ID NO: 26 - DNA, 17 bp, Corynebacterium XIV
ctgcagtcccacagtta

SEQ ID NO: 27 - DNA, 18 bp, Peptostreptococcus IV
gcatttccgcctgcgaac

SEQ ID NO: 28 - DNA, 18 bp, Peptostreptococcus V
gcattgccgcctgcgaac

SEQ ID NO: 29 - DNA, 18 bp, Peptostreptococcus VI
  misc_feature (13)..(13): n = t or g
cactatatagctnccctc

SEQ ID NO: 30 - DNA, 18 bp, Sporomusa-Gruppe
catctcagcgtcagacac

(19) United States
(12) Patent Application Publication
(10) Pub. No.: US 2005/0202477 A1
(43) Pub. Date: Sep. 15, 2005

(54) Oligonucleotides for detecting microorganisms

(76) Inventors: Andrea Saettler, Duesseldorf (DE); Claudia Jassoy, Duesseldorf (DE); Regine Scholtyssek, Mettmann (DE); Vera Maienschein, Bruchkoebel (DE); Silke Nieveler, Moenchengladbach (DE); Albrecht Weiss, Langenfeld (DE); Karl-Heinz Trebesius, Bad Endorf (DE); Claudia Beimfohr, Muenchen (DE); Wolfgang Ludwig, Sachsenkam (DE); Richard Robert Bamberg, Bruckmuehl (DE); Karl-Heinz Schleifer, Unterschleissheim (DE); Stefan V. Muellner, Langenfeld (DE); Ingrid Bergmaier, Muenchen (DE)

Correspondence Address: WOODCOCK WASHBURN LLP, ONE LIBERTY PLACE, 46TH FLOOR, PHILADELPHIA, PA, 19103, US

(21) Appl. No.: 11/039,026

(22) Filed: Jan. 18, 2005

Related U.S. Application Data
(63) Continuation of application No. PCT/EP03/07717, filed on Jul. 16, 2003.

(30) Foreign Application Priority Data
Jul. 18, 2002 (DE) 102 32 776.9
Feb. 14, 2003 (DE) 103 07 732.4

(51) Int. Cl. C12Q 1/68; C07H 21/04

(52) U.S. Cl. 435/6; 536/24.1

(57) ABSTRACT

The present invention describes oligonucleotides for detecting microorganisms, and processes and kits including the same.

### CROSS-REFERENCE TO RELATED APPLICATIONS

This application is a continuation of PCT/EP2003/007717, filed Jul. 16, 2003, which claims priority to DE 102 32 776.9, filed Jul. 18, 2002, and DE 103 07 732.4, filed Feb. 14, 2003, the disclosures of which are incorporated herein in their entireties.

### FIELD OF THE INVENTION

The present invention relates to oligonucleotides for detecting microorganisms, and processes and kits including the same.

### BACKGROUND

In the past, the detection of microorganisms mainly has been carried out serologically or microscopically.

Detection has hitherto been preceded by a cultivation step in which the microorganisms are multiplied because previous methods were not sensitive enough for the direct detection of small quantities of microorganisms. One disadvantage of the requirement for cultivation is that some of the microorganisms do not grow on the nutrient media available, and so are not detected. In fact, analyses of various environmental samples show that, at present, only 0.1 to 14% of all bacteria can be cultivated. In particular, cultivation-dependent processes have proved to be unsuitable for analysis of the composition of a complex biocoenosis. This is because, depending on the cultivation conditions selected, only those microorganisms which are particularly well adapted to the cultivation conditions proliferate, with the result that the population ratios prevailing in the starting sample are heavily distorted. Quantitative analysis of the microorganisms is totally impossible on account of such population shifts. Another disadvantage of the requirement for cultivation is that some of the known cultivation processes are very laborious and are often equivocal in their results. This leads both to false-positive and to false-negative analysis results. In view of the described disadvantages of cultivation, modem methods of microorganism detection all have a common goal: they seek to avoid the disadvantages of cultivation by eliminating the need for cultivation.

DNA-based or RNA-based hybridization or amplification methods (DNA=deoxyribonucleic acid, RNA=ribonucleic acid) can be used for detecting microorganisms. Hybridization is understood in particular to be the formation of a double helix of two single-stranded complementary oligo- or polynucleotides. Hybridization can occur in particular both between two DNA or two RNA molecules and between DNA and RNA molecules. The various molecules only hybridize when the target sequences are sufficiently complementary to one another. The complementary target sequences for the detection may also be immobilized, as is often done on so-called DNA chips. Utility Model DE 201 10 013 claims one such carrier (DNA chip) for the diagnosis and treatment of oral diseases, especially parodontitis. Oligonucleotides complementary to known reference sequences of certain bacteria or viruses occurring in the oral flora are immobilized on this carrier. By virtue of the complementarity, the oligonucleotides applied to this gene chip are able to hybridize with the corresponding reference sequences under certain conditions. The disadvantage of this carrier is that the microorganisms either have to be multiplied by cultivation or the genetic information from the samples present has to be amplified on the chip before hybridization. Accordingly, the microorganisms originally present in a sample cannot be quantified either.

Another known amplification method is the polymerase chain reaction (PCR). In the PCR, a characteristic piece of the particular microorganism genome is amplified with specific primers. If the primer finds its target site, a piece of the genetic substance undergoes a millionfold proliferation. A qualitative evaluation can be made in the subsequent analysis, for example using an agarose gel that separates DNA fragments. In the most simple case, this provides the information that the target sites for the primers used were present in the analysis sample. No other information can be provided. These target sites may originate both from a living bacterium and from a dead bacterium or from naked DNA. Differentiation is not possible here. In addition, various substances present in the analysis sample can induce inhibition of the DNA-amplifying enzyme, taq-polymerase. This is a common cause of false-negative results. A further development of the PCR technique is quantitative PCR which seeks to establish a correlation between the quantity of microorganisms present and the quantity of amplified DNA. Advantages of PCR include its high specificity and the short time it takes. Major disadvantages are its high susceptibility to contamination and the resulting false-positive results, the above-mentioned impossibility of distinguishing between living and dead cells or naked DNA and, finally, the danger of false-negative results due to the presence of inhibitory substances.

A more useful process, using in situ hybridization with fluorescence-marked oligonucleotides was developed at the beginning of the nineties and has been successfully used in many environmental samples (Amann et al. (1990), J. Bacteriol. 172, 762). The process was named “FISH” (fluorescence in situ hybridization) and makes use of the fact that the ribosomal ribonucleic acids (RNAs) occurring in every cell have both highly preserved and variable, i.e. genus- or even species-specific, sequences. Complementary oligonucleotides can be produced against these sequence domains and can be additionally provided with a detectable marker. Using these so-called nucleic acid probes, microorganism species, genera or groups can be directly identified in the sample with high specificity and, if necessary, may even be visualized or quantified.

This method is the only method which provides a distortion-free representation of the actual in situ conditions of the biocoenosis. Even hitherto non-cultivated and hence undescribed microorganisms can be identified.

In FISH, probes penetrate into the cells present in the analysis sample. If a microorganism of the species, genus or group for which the probes were developed is present in the analysis sample, the probes in the microorganism cell bind to their target sequence and the cells can be detected through the marking of the probes. The advantages of the FISH technique over the methods described further above for microorganism identification (cultivation, PCR) are many and various. Firstly, numerous microorganisms that are impossible to detect by traditional cultivation can be detected with probes. Whereas at most only 15% of the bacterial population of a sample can be made visible by cultivation, the FISH technique enables up to 100% of the total bacterial population to be detected in many samples. Secondly, microorganisms can be detected far more quickly by the FISH technique than by cultivation. Whereas the identification of microorganisms by cultivation often takes several days, only a few hours elapse between sampling and microorganism identification, even at species level, in the FISH technique. Thirdly, in contrast to a cultivation medium, probes can be almost freely selected in their specificity. Individual species can be detected just as well with a probe as entire genera or microorganism groups. Fourthly, microorganism species or entire microorganism populations can be exactly quantified in the sample itself. Fifthly, associations of various microorganisms in a sample can be visualized.

In contrast to PCR, FISH reliably detects only living microorganisms. The false-positive results through naked DNA or dead microorganisms obtained with PCR do not occur in FISH. In addition, false-negative results through the presence of inhibitory substances are ruled out as much as false-positive results attributable to contaminations. Accordingly, the FISH technique is an excellent tool for directly detecting microorganisms in a sample both rapidly and with high specificity. In contrast to cultivation processes, it is direct and, in addition, even enables the microorganisms present in the sample to be quantified.

Thus, there is a need for oligonucleotides suitable as nucleic acid probes. More particularly, there is a need for probes for detecting microorganisms coming into contact with human beings and/or animals, for example in foods, wastewaters, environmental samples, or from the skin surface.

### SUMMARY

Accordingly, the problem addressed by the present invention was to provide oligonucleotides for detecting microorganisms or microorganism groups. This would guarantee rapid (and optionally quantitative) determination of these microorganisms in a sample and, in addition, would safely allow detection individual species or groups of species of microorganisms, despite the simultaneous presence of other microorganisms.

### DETAILED DESCRIPTION

The present invention comprises oligonucleotides for the detection of microorganisms selected from the group consisting of:
i) oligonucleotides with the sequences shown in SEQ ID NO. 01 to 30 and
ii) oligonucleotides which correspond to the oligonucleotides under i) at least in 80%, preferably in at least 84%, more preferably in at least 90% and most preferably in 95% of the nucleotides and
iii) oligonucleotides derived from one of the oligonucleotides mentioned under i) and ii), the sequence being deleted or extended by one or more nucleotides and
iv) oligonucleotides which hybridize under stringent conditions with a sequence which is complementary to one of the oligonucleotides mentioned under i), ii) or iii).

The present invention also comprises processes for using these oligonucleotides and kits for applying the process.

In the context of the invention, the term “microorganism group” is understood to encompass at least two species of microorganisms which either belong to the same genus or have a very similar rRNA. For example, a microorganism group according to the invention may also contain all the species of a genus.

Besides the oligonucleotides with the sequences shown in SEQ ID NO. 01 to 30 and oligonucleotides which correspond to them in at least 80%, preferably in at least 84%, more preferably in at least 90% and most preferably in 95% of the nucleotides, the present invention also encompasses oligonucleotides that are derived from the oligonucleotides mentioned, being extended or deleted by one or more nucleotides.

In addition, besides the oligonucleotides with the sequences shown in SEQ ID NO. 19 to 30 and oligonucleotides which correspond to them in at least 77%, preferably in at least 83%, more preferably in at least 88% and most preferably in 94% of the nucleotides, the invention also encompasses oligonucleotides that are derived from the oligonucleotides mentioned, being extended or deleted by one or more nucleotides.

More particularly, 1 to 40, preferably 1 to 25 and more particularly 1 to 15 nucleotides may also be attached to the 3′ end and/or to the 5′ end of the oligonucleotides mentioned. According to the present invention, it is also possible to use oligonucleotides which are derived from the oligonucleotides mentioned by deletion of 1 to 7, preferably 1 to 5 and more preferably 1 to 3, for example 1 or 2, nucleotides from the sequence.

Microorganisms can be found in many places. For example, the human skin, with an area of around 2 m², is one of the largest human organs inhabited by microorganisms. In the course of evolution, a close relationship has developed between the host and its microbial inhabitants. The nutrients provided by the skin through various glands are metabolized by microorganisms. The resulting acidification of the skin's surface prevents it from being colonized by pathogenic microorganisms. However, the metabolism activity of microorganisms can also have unwanted effects. For example, the formation of body odor and dandruff and the development of various skin diseases can be attributed to the activity of microbes. For example, the yeast Malassezia is suspected of involvement in particular in flaking of the skin, for example on the head. In addition, this organism is regarded as the origin of the skin disease Pityriasis versicolor. An increased occurrence of Priopionibacterium acnes can be a sign of the development of acne, even in the early stages.

In principle, all microorganisms belong to the skin flora which can be isolated from the skin. According to Price, there are two different groups, resident flora which are likely attached to the skin, and transient flora, which are not attached to the skin (Price, P. B.: The bacteriology of the normal skin: A new quantitative test applied to a study of the bacterial flora and the disinfectant action of mechanical cleansing. J. Infect. Dis., 63:301-318, 1938). Resident flora are microorganisms which are capable of proliferating on the human skin or which, in analyses of skin samples, can regularly be found in large numbers or a high percentage. As noted above, these properties are attributable to the firm anchorage of these resident microorganisms to the skin. Transient flora are microorganisms which are not capable of proliferating on the human skin or which, in analyses, are only found irregularly and in small numbers/percentages. Theoretically, these microorganisms are free, i.e. do not adhere to skin constituents.

A fuller knowledge, above all of the resident skin flora, is important in particular in view of the search for new medicinal or cosmetic active components. In addition, interactions between various microorganisms can open up a broader understanding of the relations between healthy skin and its diseases and facilitate the development of better active principles, treatments or medicines. The selective influencing of relevant skin microorganisms by cosmetic products, such as deodorants, creams, etc., also presupposes a thorough knowledge of the structure and function of the micro-eco system which the skin is.

In one particular embodiment, the microorganisms to be detected are selected from the genera Staphylococcus, Peptostreptococcus, Propionibacterium, Corynebacterium, Veillonella, Malassezia and/or the Sporomusa taxon.

Hitherto, the microflora of the skin have only been investigated by the known cultivation methods. Owing to the above-mentioned deficiencies of those methods, only cultivatable bacteria or fungi could be detected. Examples of such species include Staphylococcus aureus, S. epidermidis, S. cohnii, S. haemolyticus, S. hominis, S. capitis, S. warneri, S. sciuri, S. schleiferi, S. intermedius, Veillonella spec., Propionibacterium acnes, Malassezia sloffiae, M. pachydermatis, M. furfur, Corynebacterium minutissimum, C. amycolatum, C. striatum and C. xerosis.

With the oligonucleotides according to the invention, these and other species of the genera mentioned may advantageously be detected not only qualitatively, but also quantitatively. This quantitative information can be of use, above all, for tests on active principles or the early diagnosis of skin diseases. In addition, the microorganism genera mentioned can also occur in other samples, for example in foods, clinical examination material or environmental samples. In the context of the present invention, “skin” is understood to be the human skin and/or animal skin or mucous membrane and the skin appendages (hairs, hair follicles, nails, glands).

In one particular embodiment, the oligonucleotide carries a detectable marker, preferably a fluorescence marker, which, more particularly, is covalently bonded to the oligonucleotide. The detectability of the completed hybridization of the oligonucleotide with the target sequence is a pre-requisite for the identification and, optionally, quantification of microorganisms. More particularly, this is often achieved by covalent bonding of a detectable marker to the oligonucleotide. The detectable markers used are often fluorescent groups, for example Cy-2, Cy-3 or Cy-5 (Amersham Life Sciences, Inc., Arlington Heights, USA), FITC (fluorescein isothiocyanate), CT (5,(6)-carboxytetramethylrhodamine-N-hydroxysuccinimide ester (Molecular Probes Inc., Eugene, USA)), TRITC (tetramethylrhodamine-5,6-isothiocyanate (Molecular Probes Inc., see above) or FLUOS (5,(6)-carboxyfluorescein-N-hydroxysuccinimide ester (Boehringer Mannheim, Mannheim, Germany). Alternatively, chemoluminescent groups or radioactive markings, for example 35S, 32P, 33P, 125J, are used.

However, detectability can also be established by coupling of the oligonucleotide to an enzymatically active molecule, for example alkaline phosphatase, acidic phosphatase, peroxidase, horse radish peroxidase, β-D-galactosidase or glucose oxidase. There are a number of known chromogens for each of these enzymes which may be reacted instead of the natural substrate to give colored or fluorescent products. Examples of such chromogens are set out in Table 1 below.

TABLE 1

| Enzymes | Chromogens |
|---|---|
| Alkaline phosphatase | 4-methylumbelliferyl phosphate (*) |
| and acidic phosphatase | bis(4-methylumbelliferylphosphate) (*) |
| | 3-O-methylfluorescein, flavone-3-disphosphate |
| | triammonium salt (*) |
| | p-nitrophenylphosphate disodium salt |
| Peroxidase | tyramine hydrochloride (*) |
| | 3-(p-hydroxyphenyl)-propionic acid (*) |
| | p-hydroxyphenethyl alcohol (*), 2,2′-azino-bis-(3- |
| | ethylbenzothiazoline sulfonic acid) (ABTS) |
| | ortho-phenylenediamine dihydrochloride |
| | o-dianisidine, |
| | 5-aminosalicylic acid |
| | p-ucresol (*) |
| | 3,3′-dimethyloxybenzidine |
| | 3-methyl-2-benzothiazoline hydrazone |
| | tetramethylbenzidine |
| Horse radish | H₂O₂ + diammonium benzidine |
| peroxidase | H₂O₂ + tetramethyl benzidine |
| β-D-Galactosidase | o-nitrophenyl-β-D-galactopyranoside |
| | 4-methylumbelliferyl-β-D-galactoside |
| Glucose oxidase | ABTS, glucose and thiazolyl blue |

*fluorescence

Finally, the oligonucleotides can be designed in such a way that another nucleic acid sequence suitable for hybridization is present at their 5′ end or 3′ end. This nucleic acid sequence again comprises ca. 15 to 1,000 and preferably 15 to 50 nucleotides. This second nucleic acid region can in turn be recognized by an oligonucleotide detectable by one of the means mentioned above.

Another possibility is to couple the detectable oligonucleotides to a hapten which may subsequently be contacted with an antibody that recognizes the hapten. Digoxigenin may be mentioned as an example of such a hapten. Other examples besides those mentioned are well-known to the expert.

More particularly, the enzymatic marker is selected from a group consisting of peroxidase, preferably horse radish peroxidase, and phosphatase, preferably alkaline phosphatase.

The present invention also relates to an oligonucleotide combination for the detection of microorganisms containing at least one and preferably two or more of the oligonucleotides mentioned.

In the context of the invention, an oligonucleotide combination is understood to be a composition containing at least one or more oligonucleotides, for example in a solution (for example a buffer solution) or mixture (for example in the freeze-dried state). In addition, the oligonucleotides may also be present separated from one another (for example in different containers) alongside one another (for example on a chip or in a kit).

In one particular embodiment, the oligonucleotide combination contains
i) at least one oligonucleotide for the specific detection of bacteria of the genus Staphylococcus selected from the group consisting of
   a) oligonucleotides with the sequences shown in SEQ ID NO. 01 to 03 and
   b) oligonucleotides which correspond to the oligonucleotides under a) as mentioned in claim 1 ii) and
   c) oligonucleotides which correspond to the oligonucleotides under a) as mentioned in claim 1 iii) and
   d) oligonucleotides which hybridize under stringent conditions with a sequence which is complementary to one of the oligonucleotides under a), b) or c), and/or
ii) at least one oligonucleotide for the specific detection of bacteria of the genus Peptostreptococcus selected from the group consisting of
   a) oligonucleotides with the sequences shown in SEQ ID NO. 04 to 06 and 27 to 29 and
   b) oligonucleotides which correspond to the oligonucleotides under a) as mentioned in claim 1 ii) and
   c) oligonucleotides which correspond to the oligonucleotides under a) as mentioned in claim 1 iii) and
   d) oligonucleotides which hybridize under stringent conditions with a sequence which is complementary to one of the oligonucleotides under a), b) or c), and/or
iii) at least one oligonucleotide for the specific detection of bacteria of the genus Corynebacterium selected from the group consisting of
   a) oligonucleotides with the sequences shown in SEQ ID NO. 07 to 12 and 19 to 26 and
   b) oligonucleotides which correspond to the oligonucleotides under a) as mentioned in claim 1 ii) and
   c) oligonucleotides which correspond to the oligonucleotides under a) as mentioned in claim 1 iii) and
   d) oligonucleotides which hybridize under stringent conditions with a sequence which is complementary to one of the oligonucleotides under a), b) or c), and/or
iv) at least one oligonucleotide for the specific detection of bacteria of the genus Veillonella selected from the group consisting of
   a) oligonucleotides with the sequences shown in SEQ ID NO. 13 to 15 and
   b) oligonucleotides which correspond to the oligonucleotides under a) as mentioned in claim 1 ii) and
   c) oligonucleotides which correspond to the oligonucleotides under a) as mentioned in claim 1 iii) and
   d) oligonucleotides which hybridize under stringent conditions with a sequence which is complementary to one of the oligonucleotides under a), b) or c), and/or
v) at least one oligonucleotide for the specific detection of bacteria of the species Propionibacterium acnes selected from the group consisting of
   a) oligonucleotides with the sequences shown in SEQ ID NO. 16 and 17 and
   b) oligonucleotides which correspond to the oligonucleotides under a) as mentioned in claim 1 ii) and
   c) oligonucleotides which correspond to the oligonucleotides under a) as mentioned in claim 1 iii) and
   d) oligonucleotides which hybridize under stringent conditions with a sequence which is complementary to one of the oligonucleotides under a), b) or c), and/or
vi) at least one oligonucleotide for the specific detection of fungi of the genus Malassezia selected from the group consisting of
   a) an oligonucleotide with the sequence shown in SEQ ID NO. 18 and
   b) oligonucleotides which correspond to the oligonucleotide under a) as mentioned in claim 1 ii) and
   c) oligonucleotides which correspond to the oligonucleotide under a) as mentioned in claim 1 iii) and
   d) oligonucleotides which hybridize under stringent conditions with a sequence which is complementary to one of the oligonucleotides under a), b) or c), and/or
vii) at least one oligonucleotide for the specific detection of microorganisms from the Sporomusa taxon selected from the group consisting of
   a) an oligonucleotide with the sequence shown in SEQ ID NO. 30 and
   b) oligonucleotides which correspond to the oligonucleotide under a) as mentioned in claim 1 ii) and
   c) oligonucleotides which correspond to the oligonucleotide under a) as mentioned in claim 1 iii) and
   d) oligonucleotides which hybridize under stringent conditions with a sequence which is complementary to one of the oligonucleotides under a), b) or c).

The oligonucleotides according to the invention enable microorganisms of the genera Staphylococcus, Peptostreptococcus, Propionibacterium, Corynebacterium, Veillonella, Malassezia and the Sporomusa taxon to be specifically detected.

Accordingly, the following combinations of one or more oligonucleotides from groups i) to vii) are possible according to the invention. By this is meant, for example, the selection of one or more oligonucleotides from one of the groups i), ii), iii), iv), v), vi) or vii).

The combinations of one or more oligonucleotides from group i) with one or more oligonucleotides from group ii) and, analogously, those from group i) with iii), i) with iv), i) with v), i) with vi) and i) with vii), ii) with iii), ii) with iv), ii) with v), ii) with vi) and ii) with vii), iii) with iv), iii) with v), iii) with vi) and iii) with vii), iv) with v), iv) with vi), iv) with vii), v) with vi), v) with vii) and vi) with vii) are encompassed by the invention.

The combinations of one or more oligonucleotides from groups i), ii) and iii) and, analogously, i) with ii) and iv); i) with ii) and v); i) with ii) and vi); i) with ii) and vii), i) with iii) and iv); i) with iii) and v); i) with iii) and vi), i) with iii) and vii); i) with iv) and v); i) with iv) and vi); i) with iv) and vii), i) with v) and vi), i) with v) and vii); ii) with iii) and iv); ii) with iii) and v); ii) with iii) and vi), ii) with iii) and vii); ii) with iv) and v); ii) with iv) and vi), ii) with iv) and vii); ii) with v) and vi), ii) with v) and vii); iii) with iv) and v); iii) with iv) and vi), iii) with iv) and vii); iii) with v) and vi), iii) with v) and vii) and also iv) with v) and vi), iv) with v) and vii), iv) with vi) and vii), v) with vi) and vii) are also possible in accordance with the invention.

Combinations of one or more oligonucleotides selected from each of four groups, i.e. from group i) with ii), iii) and iv); i) with ii), iii) and v); i) with ii), iii) and vi), i) with ii), iii) and vii); i) with ii), iv) and v); i) with ii), iv) and vi); i) with ii), iv) and vii), i) with ii), v) and vi), i) with ii), v) and vii); i) with ii), vi) and vii); i) with iii), iv) and v), i) with iii), iv) and vi); i) with iii), iv) and vii); i) with iii), v) and vi); i) with iii), v) and vii); i) with iv), v) and vi), i) with iv), v) and vii); i) with iv), vi) and vii); ii) with iii), iv) and v); ii) with iii), iv) and vi), ii) with iii), iv) and vii); ii) with iii), v) and vi), ii) with iii), v) and vii) or iii) with iv), v) and vi), iii) with iv), v) and vii), iii) with iv), vi) and vii), may also be used.

The combinations of one or more oligonucleotides from five groups, i.e. i) with ii), iii), iv) and v), i) with ii), iii), iv) and vi), i) with ii), iii), iv) and vii), i) with ii), iii), v) and vi), i) with ii), iii), v) and vii), i) with iii), iv), v) and vi), i) with iii), iv), v) and vii), i) with ii), iv), v) and vi), i) with ii), iv), v) and vii), i) with ii), iv), vi) and vii), ii) with iii), iv), v) and vi), ii) with iii), iv), v) and vii) are also encompassed by the invention.

The combinations of one or more oligonucleotides from six groups, i.e. i) with ii), iii), iv), v) and vi), i) with ii), iii), iv), v) and vii), i) with iii), iv), v), vi) and vii); ii) with iii), iv), v), vi) and vii), and the combination of one or more oligonucleotides from all seven groups are also covered by the invention.

Accordingly, the oligonucleotide combination according to the invention is suitable for detecting a microorganism species or a microorganism group. To this end, one or more of the oligonucleotides under i) are selected, for example, for the detection of certain species of Staphylococcus.

In addition, however, the detection of species and/or groups of microorganisms of the various genera mentioned can advantageously be carried out simultaneously and/or alongside one another by suitable composition of the oligonucleotide combination (according to the possible combinations mentioned).

For example, with a suitable combination of oligonucleotides, the detection of microorganisms of the genus Staphylococcus (by selecting one or more of the oligonucleotides under i)) may be carried out at the same time as and/or alongside the detection of microorganisms of the genus Propionibacterium acnes (by selecting one or more of the oligonucleotides under v)). The possible combinations can thus be individually adapted to meet particular requirements.

So far as the choice of the oligonucleotides for detecting microorganisms is concerned, it is particularly important that a suitable complementary sequence is present in the microorganism to be detected. A sequence is suitable when, on the one hand, it is specific to the microorganism to be detected and, on the other hand, is actually accessible to the penetrating oligonucleotide, i.e. is not masked, for example, by ribosomal proteins or rRNA secondary structures. Even Fuchs et al. (B. M. Fuchs, G. Wallner, W. Besiker, I. Schwippi, W. Ludwig and R. Amann: Flow cytometric analysis of the in situ accessibility of Escherichia coli 16S rRNA for fluorescently labeled oligonucleotide probes. Appl. Environ. Microbiol. 1998, 64 (12): 4973-4982) were able to show that a number of oligonucleotides developed on the basis of primary sequence data can only be used to a limited extent, if at all, for in situ hybridization. The covering of potential oligonucleotide binding sites by rRNA secondary structure motifs or ribosomal proteins is cited as the reason for the unsatisfactory binding behavior of the oligonucleotides mentioned. Such inaccessible regions are different for each organism and have to be re-discovered for each microorganism. Accordingly, the sequence for an oligonucleotide with good binding behavior is in no way revealed, even to an expert, from the primary sequence of the rRNA and, hence, also cannot be derived via consensus sequence such programs.

By selecting a particular sequence in accordance with the invention, it is possible to detect a microorganism species, a microorganism genus or a microorganism group. In the case of an oligonucleotide of 15 nucleotides, complementarity should exist over 100% of the sequence. With oligonucleotides comprising more than 15 nucleotides, one to several mispairing sites are allowed.

More particularly, the invention provides oligonucleotides for the specific detection of microorganisms of the genus Staphylococcus, the oligonucleotides being complementary to the rRNA and being selected from a group consisting of oligonucleotides with the sequences shown in SEQ ID NO. 01 to 03.

Each of the oligonucleotides mentioned detects at least one of the following species of the genus Staphylococcus: S. aureus, S. epidermidis, S. saccharolyticus, S. caprae, S. capitis, S. warneri, S. pasteuri, S. arlettae, S. gallinarum, S. cohnii, S. succinus, S. kloosii, S. saprophyticus, S. equorum, S. xylosus, S. haemolyticus, S. hominis, S. lugdunensis, S. chromogenes, S. auricularis, S. schleiferi, S. sciuri, S. lentus, S. vitulus, S. pulveri, S. felis, S. hyicus, S. piscifermentans, S. carnosus, S. simulans, S. intermedius, S. delphini, S. muscae and S. condimenti.

Microorganisms which have a similar rRNA sequence, but which do not belong to the genus Staphylococcus, are advantageously not detected by these oligonucleotides: Paenibacillus polymyxa, Bacillus lentus, Bacillus cereus, Bacillus subtilis, Bacillus mycoides, Proteus vulgaris, Burkholderia cepacia, Bacteroides uniformis and Pediococcus damnosus. This is a particular advantage and shows the high specificity of the probes.

The oligonucleotide with the sequence shown in SEQ ID NO. O₂ is suitable for the detection of microorganisms of the genus Staphylococcus, more particularly S. intermedius, S. delphini, S. muscae, S. condimenti, S. piscifermentans, S. carnosus, S. schleiferi, S. felis and S. simulans, preferably S. intermedius and S. schleiferi.

A combination of the oligonucleotides having the sequences shown in SEQ ID NO. 01 to 02 is particularly preferred. This combination detects at least the following species of the genus Staphylococcus: S. aureus, S. epidermidis, S. caprae, S. capitis, S. warneri, S. pasteuri, S. arlettae, S. gallinarum, S. cohnii, S. succinus, S. kloosii, S. saprophyticus, S. equorum, S. xylosus, S. haemolyticus, S. hominis, S. lugdunensis, S. chromogenes, S. auricularis, S. schleiferi, S. sciuri, S. lentus, S. vitulus, S. pulveri, S. intermedius, S. delphini, S. felis, S. muscae, S. condimenti, S. piscifermentans, S. carnosus and S. simulans.

More particularly, the invention also provides oligonucleotides for the specific detection of microorganisms of the genus Peptostreptococcus, the oligonucleotides being complementary to the rRNA and being selected from a group consisting of oligonucleotides with the sequences shown in SEQ ID NO. 04 to 06 and 27 to 29.

According to the latest knowledge, the bacteria known by the generic name of “Peptostreptococcus” may be assigned to various sub-groups, more particularly the genera Anaerococcus, Peptoniphilus and Finegoldia.

Each of the oligonucleotides mentioned detects at least one of the following species of the genera Anaerococcus, Peptoniphilus and Finegoldia known collectively as “Peptostreptococcus”: P. assaccharolyticus, P. lacrimalis, P. hareii, F. magnus, A. tetradius, A. hydrogenalis, A. lactolyticus, A. octavius and A. vaginalis.

The oligonucleotides with the sequences shown in SEQ ID NO. 04 to 06 are particularly preferred. These oligonucleotides each detect at least the following species of Peptostreptococci, more particularly those of the genus Anaerococcus: Anaerococcus hydrogenalis, A. lactolyticus, A. octavius, A. prevotii, Anaerococcus tetradius and A. vaginalis.

The species of the genus Peptostreptococcus mentioned below and other microorganisms which have a similar rRNA sequence, but which do not belong to the genus Peptostreptococcus, more particularly Anaerococcus, are advantageously not detected: Peptoniphilus lacrimalis, Peptostreptococcus anaerobius, Finegoldia magnus and Ruminococcus productus, Brevibacterium epidermidis, Abiotropha elegans and Clostridium hastiforme.

The oligonucleotide with the sequence shown in SEQ ID NO. 04 is particularly preferred. This oligonucleotide detects at least the following species of the microorganisms known by the generic name of “Peptostreptococcus”: Anaerococcus hydrogenalis, A. lactolyticus, A. octavius, A. prevotii and A. vaginalis.

More particularly, the invention additionally provides oligonucleotides for the specific detection of microorganisms of the genus Peptostreptococcus, the oligonucleotides being complementary to the rRNA and being selected from a group consisting of oligonucleotides with the sequences shown in SEQ ID NO. 27 to 29.

The species of the genus Peptostreptococcus mentioned below and other microorganisms which have a similar rRNA sequence, but which do not belong to the Peptostreptococci, are advantageously not detected: Micromonas micros, Helcococcus kunzii, Helcococcus ovis.

The oligonucleotides with the sequences shown in SEQ ID No. 27 and 28 are particularly preferred. These oligonucleotides detect at least the following species of the genus Peptoniphilus: Peptoniphilus assaccharolyticus, P. hareii, P. indolicus (more particularly the strain ATCC 29427 and closely related strains, i.e. strains with a very similar rRNA) and P. lacrimalis.

The following species with a similar rRNA are not detected by these oligonucleotides: Pseudomonas saccharophila, Variovorax paradoxus, Finegoldia magna, Staphylococcus epidermidis, Propionibacterium acnes, Micromonas micros, Gallicola baranese, Atopobium parvulum, Veillonella dispar, Pseudomonas putida and species of the genera Anaerococcus and Corynebacterium.

The oligonucleotide with the sequence shown in SEQ ID NO. 28 detects in particular microorganisms of the species Peptoniphilus lacrimalis.

The oligonucleotide with the sequence shown in SEQ ID NO. 29 is also particularly preferred. From the microorganisms known generically as “Peptostreptococci”, this oligonucleotide detects at least the species Finegoldia magna and also microorganisms very similar to this species in their rRNA sequence, whereas the following microorganisms cannot be simultaneously detected: Anaerococcus hydrogenalis, Peptostreptococcus anaerobius, Peptoniphilus lacrimalis, Staphylococcus epidermidis, Halocella cellulosilytica, Propionibacterium acnes, Micromonas micros, Veillonella dispar, Pseudomonas putida and other species of the genera Anaerococcus, Corynebacterium and Peptoniphilus.

More particularly, the invention provides oligonucleotides for the specific detection of microorganisms of the genus Corynebacterium, the oligonucleotides being complementary to the rRNA and being selected from a group consisting of oligonucleotides with the sequences shown in SEQ ID NO. 07 to 12.

Each of the oligonucleotides mentioned detects at least one of the following species of the genus Corynebacterium: C. glutamicum, C. lipophiloflavum, C. glucuronolyticum, C. macginleyi, C. accolens, C. fastidiosum, C. segmentosum, C ammoniagenes, C. minutissimum, C. flavescens, C. coyleiae, C. afermentans, C. pseudogenitalium, C. genitalium, C. mucofaciens, C. auris, C. mycetoides, C. cystitidis, C. pilosum, C. pseudotuberculosis, C. ulcerans, C. diphteriae, C. vitarumen, C. kutscheri, C. genitalium, C argentoratens, C. callunae, C bovis, C. variabilis, C. amycolatum, C. “tuberculostearicum”, C. xerosis, C. matruchotii, C. jeikeium, C. efficiens, C. thomsenii, C. nigricans, C. auriscanis, C. mooreparkense, C. casei, C. camporealensis, C. sundsvallense, C. mastidis, C. imitans, C. riegelii, C. asperum, C. freneyi, C. striatum, C. coyleiae and C. simulans.

Microorganisms which have a similar rRNA sequence, but which do not belong to the genus Corynebacterium, are advantageously not detected by these oligonucleotides: Clostridium acetobutylicum, Eubacterium moniliforme and Fusobacterium nucleatum. The following bacteria which belong to the skin microflora are also not detected: Micrococcus luteus, Micrococcus varians, Micrococcus lyae, Acinetobacter calcoaceticus and Streptococcus pyogenes. This is a particular advantage and shows the high specificity of the probes.

The oligonucleotide with the sequence shown in SEQ ID NO. 10 is particularly preferred for the detection of Corynebacteria of the species C. striatum and/or C. xerosis.

In addition, the oligonucleotide with the sequence shown in SEQ ID NO. 11 is used for the detection of Corynebacteria of the species C. jeikeium.

A combination of the oligonucleotides having the sequences shown in SEQ ID NO. 07, 08, 10 and 11 is particularly preferred. This combination detects at least the following species of the genus Corynebacterium: C. glutamicum, C. lipophiloflavum, C. glucuronolyticum, C. macginleyi, C. accolens, C. fastidiosum, C. segmentosum, C. ammoniagenes, C. minutissimum, C. flavescens, C. coyleiae, C. afermentans, C. pseudogenitalium, C. “genitalium”, C. mucofaciens, C. auris, C. mycetoides, C. cystitidis, C. pilosum, C. pseudotuberculosis, C. ulcerans, C. diphteriae, C. camporealensis, C. vitarumen, C. kutscheri, C. argentoratens, C. callunae, C. bovis, C. renale, C. riegelii, C. C. variabilis, C. amycolatum, C. “tuberculostearicum”, C. xerosis, C. matruchotii, C. jeikeium.

In one particular embodiment, the invention provides oligonucleotides for the specific detection of microorganisms of the genus Corynebacterium, the oligonucleotides being complementary to the rRNA and being selected from a group consisting of oligonucleotides with the sequences shown in SEQ ID NO. 19 to 26.

Each of the oligonucleotides mentioned detects at least one of the following species of the genus Corynebacterium: C. coyleiae, C. afermentans, C. “genitalium”, C. mucifaciens, C. amycolatum, C. “tuberculostearicum” and C. riegelii. These oligonucleotides are suitable for the specific detection of a group of one or more very closely related species of the genus Corynebacterium.

The following microorganisms with a similar rRNA sequence are advantageously not detected by these oligonucleotides: Clostridium acetobutylicum, Eubacterium yurii and Fusobacterium nucleatum. The following bacteria which belong to the skin microflora are also not detected: Micrococcus luteus, Micrococcus varians, Micrococcus lyae, Acinetobacter calcoaceticus and Streptococcus pyogenes. This is a particular advantage and shows the high specificity of the probes.

In a particularly preferred embodiment, the oligonucleotide with the sequence shown in SEQ ID NO. 19 is used for the detection of a group of microorganisms from the genus Corynebacterium which is formed by C. “tuberculostearicum” (more particularly ATCC 35692) or the group around the strain with the name CDC G5840 (Acc. No. X80498) and microorganisms which have a very similar rRNA, i.e. microorganisms which are very closely related to the microrganism or whose rRNA has a high degree of sameness and/or corresponds completely or almost completely (i.e. with a deviation of one or more, preferably one to three nucleotides) with the rRNA of the microorganisms mentioned in the section hybridizing with the oligonucleotide mentioned.

This probe advantageously detects C. “tuberculostearicum” and the species of the genus Corynebacterium which have a very similar rRNA without detecting the following, more distantly related species of the genus Corynebacterium: C. minutissimum, C diphteriae, C. striatum, C. xerosis, C. “fastidiosum”, C. camporealensis, C. accolens und C. “pseudogenitalium” and C. afermentans, C. jeikeium, C. durum, C. mucifaciens, C. renale, C. riegelii, C. glutamicum, C lipophiloflavum, C glucuronolyticum C. ammoniagenes, C. coyleiae, C. pseudotuberculosis, C kutscheri, C. callunae and C. urealyticum.

The probe with the sequence shown in SEQ ID NO. 20 is particularly preferred for the specific detection of C. amycolatum and closely related species. This probe advantageously detects C. amycolatum and species of the genus Corynebacterium which have a very similar rRNA and which have only a few mispairings, preferably no mispairings, in the section of the rRNA hybridizing with the oligonucleotide mentioned without detecting the following, more distantly related species of the genus Corynebacterium: C. “asperum”, C. jeikeium, C. bovis, C. freneyi, C. afermentans, C. durum, C. matruchotii, C. mucifaciens, C. renale, C. glutamicum and C. xerosis and also C. lipophiloflavum, C. glucuronolyticum, C. minutissimum, C. ammoniagenes, C. camporealensis, C. coyleiae, C. pseudotuberculosis, C. riegelii, C. kutscheri, C. callunae and C. urealyticum.

The oligonucleotide with the sequence shown in SEQ ID NO. 21 is particularly preferred for the detection of certain species of microorganisms, more particularly of the genus Corynebacterium, which correspond to the partial sequence of the 16 S rRNA shown in SEQ ID NO. 31 in at least 60%, preferably in at least 70%, more preferably in at least 80% and most preferably in at least 90%, for example at least 95%, of the nucleotides.

This probe advantageously detects the above-mentioned species of the genus Corynebacterium without detecting the following, more distantly related species of the genus Corynebacterium: C. “genitalium”, C. mucifaciens, C. coyleiae, C. glucuronolyticum, C. afermentans, C. pseudogenitalium and C. lipophiloflavum and also C. amycolatum, C. jeikeium, C. durum, C. renale, C. striatum, C. glutamicum, C. accolens, C. xerosis, C. minutissimum, C. camporealensis, C. coyleiae, C. pseudotuberculosis, C. kutscheri, C. callunae and C. urealyticum.

The oligonucleotide with the sequence shown in SEQ ID NO. 23 is particularly preferred for the detection of Corynebacteria of the species C. afermentans.

This probe advantageously detects C. afermentans and species of the genus Corynebacterium with a very similar rRNA without detecting the following, more distantly related species of the genus Corynebacterium: C. “genitalium”, C. mucifaciens, C. ammoniagenes, C. coyleiae, C. glucuronolyticum, C. riegelii, C. thomssenii. C. pseudogenitalium and C. lipophiloflavum and also C. amycolatum, C. jeikeium, C durum, C. renale, C. striatum, C. glutamicum, C. accolens, C. xerosis, C. minutissimum, C. camporealensis, C. coyleiae, C. pseudotuberculosis, C. kutscheri, C. callunae and C. urealyticum.

The oligonucleotide with the sequence shown in SEQ ID NO. 25 is particularly preferred for the detection of Corynebacteria of the species C. afermentans, C. mucifaciens, C. coyleiae and/or “C. genitalium”.

This probe advantageously detects C. afermentans, C. mucifaciens, C. coyleiae and “C. genitalium” and species of the genus Corynebacterium which have a very similar rRNA without detecting the following, more distantly related species of the genus Corynebacterium: C. xerosis, C. jeikeium, C. urealyticum, C. amycolatum, C. glutamicum, C. striatum, C. accolens, C. renale, C. ammoniagenes and C. kutscheri and also C. glucuronolyticum, C. camporealensis, C. pseudotuberculosis, C. durum, C. minutissimum, C. lipophiloflavum, C. callunae and C. thomssenii.

In addition, this oligonucleotide also does not detect the following microorganisms which, although not belonging to the genus Corynebacterium, do have a very similar rRNA: Nanomurea fastidiosa, Micromonospora echinospora, Abiotropha elegans and Arcanobacterium pyogenes.

The probe with the sequence shown in SEQ ID NO. 26 is particularly preferred for the specific detection of C. riegelli.

In another particular embodiment, the invention additionally provides oligonucleotides for the specific detection of microorganisms of the genus Veillonella, the probes being complementary to the rRNA and being selected from a group consisting of oligonucleotides with the sequences shown in SEQ ID NO. 13 to 15.

Each of the oligonucleotides mentioned detects at least one of the following species of the genus Veillonella: V. dispar, V. parvula and V. atypica. Since the genus Veillonella is largely isolated in the phylogenetic tree, non-target organisms are advantageously not detected.

A combination of the oligonucleotides having the sequences shown in SEQ ID NO. 13 to 14. This combination detects at least the following species of the genus Veillonella: V. dispar, V. parvula and V. atypica.

In a particular embodiment, the invention additionally provides oligonucleotides for the specific detection of microorganisms of the species Propionibacterium acnes, the probes being complementary to the rRNA and being selected from a group consisting of oligonucleotides with the sequences shown in SEQ ID NO. 16 to 17. Each of the oligonucleotides mentioned specifically detects the species Propionibacterium acnes. The oligonucleotide with the sequence shown in SEQ ID NO. 16 is particularly preferred.

Microorganisms which have a similar rRNA sequence, but which do not belong to the species Propionibacterium acnes, are advantageously not detected: P. propionicus, P. granulosum, P. avidum, P. freudenreichii, P. thoeni, P. lymphophilus, C. minutissimum, Saccharomonospora viridis, Nocardiodes spec., Propioniferax innocua, Gordonia sputi and Arcanobacterium.

In another particular embodiment, the invention additionally provides an oligonucleotide for the specific detection of microorganisms of the genus Malassezia, the oligonucleotide being complementary to the rRNA and having the sequence shown in SEQ ID NO. 18.

The oligonucleotide mentioned detects at least one of the following species of the genus Malassezia: M. sloffiae, M. pachydermatis, M. furfur.

Microorganisms which have a similar rRNA sequence, but which do not belong to the genus Malassezia, are advantageously not detected: Candida albicans and Candida krucei.

The oligonucleotide with the sequence shown in SEQ ID NO. 30 is particularly preferred for the detection of certain microorganisms of the Sporomusa taxon, preferably the microorganisms of the genera Phascolarctobacterium and Acidaminococcus which form a sub-group of the Sporomusa taxon and microorganisms which have a very similar rRNA to the microorganisms mentioned.

The oligonucleotide mentioned detects at least the species Acidaminococcus fermentans, Phascolarctobacterium faecium and closely related microorganisms with a very similar rRNA, but not the following microorganisms: Veillonella spec. Halobacillus halophilus, Sporomusa paucivorans, Macrococcus caseolyticus, Anaeromusa acidaminophila, Halocella cellulosilytica, Peptostreptococcus anaerobius, Succiniclasticum ruminis and Succinispira mobilis.

In one particularly preferred embodiment, unmarked oligonucleotides may be used together with marked oligonucleotides. The incubation of samples containing both unmarked and marked oligonucleotides is preferably used to increase the specificity of the probes. For example, closely related species of microorganisms may be differentiated by using—for a microorganism species not to be detected closely related to a species to be detected—an oligonucleotide which hybridizes better with the target sequence of the rRNA of the microorganism not to be detected than the marked probe under the selected conditions. Since the unmarked probe hybridizes better with the rRNA of the microorganism not to be detected than the marked probe, binding of the marked probe to the rRNA of the microorganism not to be detected and, hence, a false-positive result are prevented by the use of the unmarked oligonucleotide (competitor). The specific detection of certain microorganism species or microorganism groups is thus possible, above all even in the presence of closely related species with a very similar rRNA sequence.

For example, it is suitable in accordance with the invention to use the oligonucleotide according to SEQ ID NO. 22 together with the oligonucleotide according to SEQ ID NO. 21. In this case, the oligonucleotide with the SEQ NO. ID 21 is preferably marked and the oligonucleotide with the SEQ ID NO. 22 unmarked. The microorganism species of which the 16 S rRNA sequence comprises the sequence shown in SEQ ID No. 31 can therefore be detected without difficulty without C. afermentans being detected at the same time (cf. analysis result in the Example).

It can also be suitable in accordance with the invention to use oligonucleotides with the SEQ ID NO. 23 and 24 together. Whereas the oligonucleotide with the SEQ ID NO. 23 is used marked as the probe for detecting C. afermentans, the oligonucleotide according to SEQ ID NO. 24 masks the very similar target sequence of the microorganism species of which the 16 S rRNA sequence includes the sequence shown in SEQ ID NO. 31.

In addition, the oligonucleotide according to SEQ ID NO. 26 may be used as an unmarked competitor together with the oligonucleotide according to SEQ ID NO. 25. In this way, the following species of the genus Corynebacterium close to each other in the phylogenetic tree can be detected: C. afermentans, C. genitalium, C. mucifaciens, C. coyleiae, without the Corynebacterium species C. riegelii, which has a very similar rRNA sequence, being detected at the same time.

In a preferred embodiment, the oligonucleotide combination of one or more oligonucleotides according to SEQ ID NO. 19 to 30 contains one or more other oligonucleotides for detecting species of the genera Staphylococcus, Veillonella, Malassezia and/or Propionibacterium. Various skin-relevant microorganisms may advantageously be detected at the same or “in parallel” in one sample, more particularly in a single process. In addition, oligonucleotides disclosed herein, especially those according to SEQ ID NO. 1 to 18, are particularly suitable.

The sequences of the sequence protocol are shown in Table 2 below.

TABLE 2

| | | |
|---|---|---|
| SEQ ID NO. | Sequence 5′ → 3′ | Specificity |

| | | |
|---|---|---|
| 01 | CAC ATC AGC GTC AGT TAC | Staphylococcus I |
| 02 | CAC ATC AGC GTC AGT TGC | Staphylococcus II |
| 03 | AAG CTT AAG GGT TGC GCT | Staphylococcus III |
| 04 | GCC TTC TAA ATC ACG CGG | Peptostreptococcus I |
| 05 | AGC CCA AGT CAT AAA GGG | Peptostreptococcus II |
| 06 | TAC ACT CTC TCA AGC CGG | Peptostreptococcus III |
| 07 | AGC ACT CAA GTT ATG CCC | Corynebacterium I |
| 08 | AGT ACT CAA GTT ATG CCC | Corynebacterium II |
| 09 | AGC ACT CAA GTA ATG CCC | Corynebacterium III |
| 10 | AGC ACT CAA GTC A-G CCC | Corynebacterium IV |
| 11 | AGC ACT CTA GTT ATG CCC | Corynebacterium V |
| 12 | GGC CGG CTT TCA GCG ATT | Corynebacterium VI |
| 13 | GCT TCC ATC GCT CTT CGT | Veillonella I |
| 14 | GTT CTG TCC ATC AAT GTC | Veillonella II |
| 15 | TTC CGT CTA TTA ACT CCC | Veillonella III |
| 16 | TCA CGC TTC GTC ACA GGC | Propionibacterium acnes |
| 17 | CAG GCT CGC CAC TCT CTG | Propionibacterium acnes |
| 18 | TAC GGC GAT TCC AAA AAC C | Malassezia |
| 19 | CAC ACT AAA AAT GGC TCC | Corynebacterium VII |
| 20 | TCC ACA CCA TGG TCC TAT | Corynebacterium VIII |
| 21 | CCA TCC AAA ATG CGG TCC | Corynebacterium IX |
| 22 | CCA TCC AAA ATG TGG TCC | Corynebacterium X |
| 23 | CAC CAT CCA AAA TGT GGT C | Corynebacterium XI |
| 24 | CAC CAT CCA AAA TGC GGT C | Corynebacterium XII |
| 25 | CTG CAG TCC CGC AGT TA | Corynebacterium XIII |
| 26 | CTG CAG TCC CAC AGT TA | Corynebacterium XIV |
| 27 | GCA TTT CCG CCT GCG AAC | Peptostreptococcus IV |
| 28 | GCA TTG CCG CCT GCG AAC | Peptostreptococcus V |
| 29 | CAC TAT ATA GCT T/GCC CTC | Peptostreptococcus VI |
| 30 | CAT CTC AGC GTC AGA CAC | Sporomusa-Gruppe |

In one embodiment, a process according to the present invention comprises the following steps:
a) taking a sample,
b) fixing the microorganisms present in the sample taken,
c) incubating the fixed microorganisms with at least one oligonucleotide to induce hybridization
d) removing non-hybridized oligonucleotides and
e) detecting and optionally quantifying the microorganisms hybridized with the oligonucleotides.

In the context of the present invention, “fixing” of the microorganisms is understood to be a treatment by which the microorganism cell wall is made permeable to oligonucleotides. Ethanol is normally used for fixing. If the cell wall cannot be penetrated by the oligonucleotides following these measures, enough other measures leading to the same result are known to the expert. These include, for example, methanol, mixtures of alcohols, a low-percentage paraformaldehyde solution or a dilute formaldehyde solution or the like.

According to the invention, the fixed cells are incubated with, in particular, fluorescence-marked oligonucleotides for “hybridization”. These marked oligonucleotides are capable of binding themselves to the target sequence corresponding to the oligonucleotide, optionally after penetrating the cell wall. Binding is understood to be the development of hydrogen bridges between complementary nucleic acid fragments.

The oligonucleotides are used with a suitable hybridization solution in the process according to the invention. Suitable compositions of this solution are well-known to the expert. A corresponding solution contains, for example, formamide in a concentration of 0% to 80%, preferably 0% to 45% and more particularly 20% to 40% and, for example, has a salt concentration (the salt is preferably NaCl) of 0.1 mol/l to 1.5 mol/l, preferably 0.5 mol/l to 1.0 mol/l and more particularly 0.9 mol/l. In addition, a detergent (generally SDS) is generally present in a concentration of 0.001% to 0.2%, preferably 0.005% to 0.1% and more particularly 0.01%. A suitable buffer substance (for example Tris-HCl, Na citrate, HEPES, PIPES, etc.) is present for buffering the solution, typically in a concentration of 0.01 mol/l to 0.1 mol/l, preferably in a concentration of 0.01 mol/l to 0.05 mol/l and more particularly in a concentration of 0.02 mol/l. The pH of the hybridization solution is generally between 6.0 and 9.0, preferably between 7.0 and 8.0 and more particularly around 8.0.

Other additives may be used including, for example, fragmented salmon sperm DNA or blocking reagents for preventing non-specific bindings in the hybridization reaction or even polyethylene glycol, polyvinyl pyrrolidone or dextran sulfate for accelerating the hybridization reaction. In addition, substances may also be added to color the DNA of all the living and/or . . . . organisms present in the sample (for example DAPI, 4′, 6-diamidino-2-phenylindole dihydrochloride). Corresponding additives are all well-known to the expert and may be added in the known and typical concentrations.

The concentration of the oligonucleotide in the hybridization solution is determined by the nature of its marking and by the number of target structures. In order to provide for rapid and efficient hybridization, the number of oligonucleotides should exceed the number of target structures by several orders of magnitude. However, it is important to bear in mind that an excessive quantity of fluorescence-marked oligonucleotides leads to increased background fluorescence. Accordingly, the concentration of oligonucleotides should be in the range from 0.5 to 500 ng/μl. The preferred concentration for the process according to the invention is 1 to 10 ng of each oligonucleotide used per μl hybridization solution. The volume of hybridization solution used should be between 8 μl and 100 ml; in a preferred embodiment of the invention, it is between 10 μl and 1,000 μl and, in a particularly preferred embodiment, between 20 μl and 40 μl.

The duration of the hybridization is normally between 10 minutes and 12 hours and preferably about 1.5 hours. The hybridization temperature is preferably between 44° C. and 48° C. and more particularly 46° C. The parameter of the hybridization temperature and also the concentration of salts and detergents in the hybridization solution can be optimized in dependence upon the oligonucleotides, more particularly their lengths and the degree of complementarity to the target sequence in the cell to be detected. The expert is familiar with calculations of relevance in this regard.

On completion of the hybridization, the non-hybridized and surplus oligonucleotides should be removed or washed off, which is normally done with a conventional washing solution. If desired, this washing solution may contain 0.001 to 0.1% of a detergent, such as SDS, a concentration of 0.01% being preferred, and Tris-HCl or another suitable buffer substance in a concentration of 0.001 to 0.1 mol/l, preferably 0.02 mol/l, the pH being in the range from 6.0 to 9.0 and preferably around 8.0. The detergent may be present, but is not absolutely essential. In addition, the washing solution normally contains NaCl in a concentration—depending on the stringency required—of 0.003 mol/l to 0.9 mol/l and preferably 0.01 mol/l to 0.9 mol/l. An NaCl concentration of 0.07 mol/l is particularly preferred. NaCl concentrations of 0.05 mol/l to 0.22 mol/l are particularly suitable for hybridizations in which specific detections can be carried out with the oligonucleotides according to SEQ ID NO. 19 to 30. In addition, the washing solution may contain EDTA in concentrations of preferably 0.005 mol/l. The washing solution may also contain preservatives known to the expert in suitable quantities.

The non-bound oligonucleotides are normally “washed off” at a temperature of 44° C. to 52° C., preferably at a temperature of 44° C. to 50° C. and more particularly at a temperature of 44° C. to 48° C. over a period of 10 to 40 minutes and preferably over a period of 15 minutes.

Depending upon the nature of the marking of the oligonucleotide used, the concluding evaluation may be carried out with a light microscope, an epifluorescence microscope, a chemoluminometer, fluorometer, etc.

The advantages of the process according to the invention are many and various.

A particular advantage is the speed of this detection process. Whereas the traditional cultivation needs up to seven days for detection, the result is available in three hours after application of the process according to the invention. This provides for the first time for the accompanying diagnostic control of the effects and unwanted effects of an applied treatment. Another advantage in this regard is that the process according to the invention enables all the microorganisms mentioned to be simultaneously detected, which is another time advantage because all steps from sampling to evaluation only have to be carried out once.

Another advantage is that the microorganisms detected can be quantified.

Another advantage is the fact that microorganisms of the skin flora, which could not be detected by traditional detection processes, can now be detected for the first time by the oligonucleotides provided by the invention.

Various groups of microorganisms can be detected according to the specificity of the oligonucleotide or oligonucleotides used. On the one hand, large groups of microorganisms and, on the other hand, relatively small, closely related groups and even individual species can be specifically detected alongside other, even closely related microorganism species.

In addition, it is possible by the process according to the invention—in the case of the positive signal—to incorporate unknown microorganism species in the phylogenetic tree or to confirm assignings undertaken on the basis of biochemical detection by way of hybridization with a specific probe.

Another advantage is the high specificity of the oligonucleotides. Thus, certain genera or groups of microorganisms may be specifically detected while individual species of a genus may be detected with high specificity.

In a preferred embodiment of the process according to the invention, the sample is taken in step a) of the process
i) from the skin surface,
ii) from foods,
iii) from the environment, particularly from water, soil or air,
iv) from wastewater or from a biofilm,
v) from clinical examination material or
vi) from a pharmaceutical or cosmetic product.

In this preferred embodiment of the process according to the invention, the sample is taken from the skin surface by removing microorganisms of the skin flora from the area to be tested with the aid of a detergent solution.

Another major advantage is that, for the first time, these medicinally and cosmetically relevant microorganisms of the skin flora can now be simultaneously detected. Thus, by using different markers for the oligonucleotides, all, several or individual microorganism groups or species can be detected in parallel and clearly differentiated from one another. In addition, the population ratios of these microorganism groups or species and the interactions between them can thus be analyzed for the first time. This opens up for the first time the possibility of unequivocally diagnosing and selectively treating medicinally and/or cosmetically relevant skin problems. It is now possible for the first time to determine the effects of a medicinal therapy or cosmetic treatment on the overall microflora of the skin. Possible effects and unwanted effects of a treatment can thus be recognized early and amplified or suppressed in the further treatment.

Another advantage is that the microorganisms detected can be quantified. Knowledge of the absolute and relative quantitative ratios of the above-mentioned microorganisms of the skin microflora can thus be acquired for the first time. This enables the outcome of a medical or cosmetic treatment and all its effects to be monitored before, during and after the treatment. Another advantage in this connection is that the process according to the invention detects only living microorganisms.

To take skin samples from the volunteer, the skin is contacted with a detergent solution which is intended to facilitate removal of the microorganisms from the skin surface. Physiologically safe detergents such as, for example, Tween or Triton in concentrations of ca. 0.01 to 1% by weight are preferably used. A pH of 5 to 10 and more particularly in the range from 7 to 9, for example 8, has proved to be favorable.

In order to achieve better removal of the microorganisms, the surface of the skin is rubbed with a scraping instrument. Suitable scraping instruments are rods varying in diameter, for example from 0.05 to 1.5 cm, of various materials such as, for example, glass, metal or plastic. Rounded spatulas of the same materials are also suitable. Glass rods between 0.4 and 0.8 cm in diameter or plastic spatulas are preferably used. Mouthpieces of glass pipettes, for example a 5 ml glass pipette, may also be used with advantage. It has proved to be particularly suitable to rub relatively rough surfaces over the skin in order to improve removal of the microorganisms.

Plastic spatulas with a rough surface, for example a sampling spatula of glass-fiber-reinforced polyamide (Merck, Art. No. 231J2412, double spatula, length 180 mm) are particularly suitable. Rubbing with swabs and sampling by dabbing with relatively viscous media or even skin sampling with adhesive film (for example commercially available household adhesive tape) are also suitable for the purposes of the invention. With these methods, the microorganisms can be obtained, for example, by washing off with a suitable buffer solution. The other process may even be carried out on the adhesive tape itself.

The process according to the invention is preferably also used in the control of foods. The food samples are taken in particular from milk or dairy products (yoghurt, cheese, whey, butter, buttermilk), drinking water, beverages (carbonated drinks, beer, juices), confectionery or meats.

In addition, environmental samples, for example, can be examined for the presence of microorganisms using the process according to the invention. These samples may be taken from air, water or from the soil.

The process according to the invention may also be used for the analysis of clinical samples. It is suitable for the examination of tissue samples, for example biopsy material from the lungs, tumor or inflamed tissue, from secretions, such as perspiration, saliva, sperm and discharges from the nose, urethra or vagina and for urine and stool samples.

Another application for the process according to the invention is the analysis of wastewaters, for example activated sludge, digested sludge or anaerobic sludge. In addition, it is suitable for analyzing biofilms in industrial plants and also naturally developing biofilms or biofilms formed in the treatment of wastewater.

The process according to the invention may also be used for analyzing pharmaceutical and cosmetic products, for example ointments, creams, tinctures, sirups, etc., for example for contamination by microorganisms.

In another preferred embodiment of the invention, fixing is carried out by i) denaturing reagents preferably selected from a group consisting of ethanol, acetone and ethanol/acetic acid mixtures, ii) crosslinking reagents preferably selected from the group consisting of formaldehyde, paraformaldehyde and glutaraldehyde or iii) as heat fixing.

In one particular embodiment, the microorganisms may be immobilized on a carrier after fixing.

In a particularly preferred embodiment, the fixed cells of the microorganisms are permeabilized before step c) of the process according to the invention.

In the context of the invention, “permeabilizing” is understood to be an enzymatic treatment of the cells. This treatment makes the cell wall of fungi and gram-positive bacteria permeable to the oligonucleotides. Enzymes suitable for this treatment, suitable concentrations thereof and suitable solvents are known to the expert. The process according to the invention is of course also suitable for the analysis of gram-negative bacteria; the enzymatic treatment for permeabilizing is then adapted accordingly or may even be omitted altogether.

Permeabilizing the cells before hybridization has the advantage that, although the oligonucleotides are able to penetrate into the cells, the ribosomes and hence the rRNA are unable to escape from the cells. The major advantage of this technique of whole-cell hybridization is that the morphology of the bacteria remains intact and these intact bacteria can be detected in situ, i.e. in their natural surroundings. Accordingly, not only can the bacteria be quantified, possible associations between various bacterial groups can also be detected.

In a most particularly preferred embodiment, permeabilizing may be carried out by partial degradation using cell-wall-lytic enzymes preferably selected from the group consisting of lysozyme, lysostaphin, proteinase K, pronase and mutanolysin.

In addition, in a particularly preferred embodiment, the present invention provides an oligonucleotide suitable as a positive control. This oligonucleotide is characterized in that it detects many, optimally all, of the bacteria or eucaryotes present in the analyzed sample. For example, the oligonucleotide EUB338 (bacteria) described by Amann et al. (1990) or the oligonucleotide EUK (eucaryotes) is suitable for this purpose. A positive control such as this may be used to monitor whether the applied process is being carried out properly. Above all, however, it enables a proportion of the microorganisms specifically detected in the bacterial population as a whole to be determined.

The invention also provides a kit for applying the process according to the invention. This kit contains the particular hybridization solutions containing the oligonucleotides specific to the microorganisms to be detected as its most important constituents. In addition, it may contain a corresponding hybridization solution without oligonucleotides and the corresponding washing solution or a concentrate of the corresponding washing solution. In addition, it may optionally contain enzyme solutions, fixing solutions and optionally an embedding solution. Hybridization solutions for simultaneously carrying out a positive control and a negative control (for example without or with non-hybridizing oligonucleotides) may optionally be present.

In one particular embodiment, the kit is used for detecting microorganisms of the skin microflora. Thus, the use of the kit is advantageous in the search for active substances, in the analysis of the skin microflora and in the effect-testing of cosmetics containing active substances. The analysis of samples both of human skin and of animal skin can be efficiently carried out with the kits according to the invention, even against a high background of other microorganisms.

A kit containing several oligonucleotides or oligonucleotide combinations is particularly suitable. In a particularly preferred embodiment, oligonucleotides or oligonucleotide combinations capable of detecting a larger group of the microorganisms to be detected are used in a kit containing one or more oligonucleotides capable of detecting only one or a few species belong to that group. For example, it may be practical first to identify samples containing microorganisms of the genus Corynebacterium using one or more probes and then to investigate the positive samples specifically for individual microorganism species or groups within the genus Corynebacterium. Preferred oligonucleotides which may preferably be used—more particularly in combination—for detecting many different species of the genus Corynebacterium, preferably for detecting the skin-relevant species of the genus Corynebacterium, are the oligonucleotides with the sequence shown in SEQ ID NO. 7 to 12, more particularly in SEQ ID NO. 7, 8, 10 and 11, or a combination thereof, more particularly the oligonucleotide combination which contains all oligonucleotides according to SEQ ID NO. 7, 8, 10 and 11. To this end, one or more of the above-mentioned oligonucleotides according to SEQ ID NO. 19 to 26 may be added to the kit, depending on the species of the interesting microorganism of the genus Corynebacterium.

The following Examples are intended to illustrate the invention without limiting it in any way.

### EXAMPLES

Detection of Microorganisms of the Skin Microflora

Sampling

Sampling is carried out by the detergent washing method ((P. Williamson, A. M. Kligman (1965), J. Invest. Derm., Vol. 45, No. 6).

Procedure:

1. The plastic cylinder open at both ends is pressed with the undamaged end onto the skin surface to be investigated and filled with 1.5 ml of the detergent solution (a physiological Tween buffer solution, pH 8.0, containing 0.523 KH₂PO₄ g/liter, 16.73 K₂HPO₄ g/liter, 8.50 NaCl g/liter, 10.00 Tween 80 g/liter and 1.00 tryptone g/liter).

2. With one of the scraping instruments described above, the area to be treated is rubbed under light pressure 6× horizontally and 6× vertically.

3. The procedure is repeated after the liquid has been removed under suction.

The two liquids are combined. Part of the sample of the two combined liquids is used for the subsequent detection using oligonucleotides; another part is used for the parallel detection—serving as control—by cultivation of the microorganisms present in the sample.

Germ-free water (for example millipore water) should be used to prepare the detergent solution.

Fixing

One volume of absolute ethanol is then added to the sample taken, followed by centrifuging (room temperature, 8,000 r.p.m., 5 minutes). The supernatant liquid is discarded and the pellet is washed in one volume of 1×PBS solution. Finally, the pellet is re-suspended in 1/10 volume of fixing solution (50% ethanol) and stored at −20° C. pending further use.

An aliquot of the cell suspension is applied to a microscope slide and dried (46° C., 30 mins. or until completely dry). The cells are then completely dehydrated by applying another fixing solution (absolute ethanol) and drying (46° C., 3 mins. or until completely dry).

Permeabilizing

A suitable volume of a suitable enzyme solution is then applied and the sample is incubated (room temperature, 15 mins.). This step is optionally repeated with another suitable enzyme solution.

The permeabilizing solution is removed with distilled water and the sample is again completely dried (incubation at 46° C. until completely dry). The cells are then completely re-dehydrated by applying the fixing solution (absolute ethanol) and drying (46° C., 3 mins. or until completely dry).

Hybridization

The hybridization solution containing the above-described oligonucleotides specific to the microorganisms to be detected is then applied to the fixed, completely digested and dehydrated cells. The slide is then placed in a chamber moistened with hybridization solution (with no nucleotides for 90 mins. at 46° C.

Washing

The microscope slide is then placed in a chamber filled with washing solution and incubated (46° C., 15 mins.).

The slide is then briefly immersed in a chamber filled with distilled water and air-dried in the lateral position (46° C., 30 mins. or until completely dry).

Detection

The specimen holder is then embedded in a suitable embedding medium. The sample is then analyzed using a fluorescence microscope.

Analysis Results

Using the sampling method described above, microorganism samples were taken from the forehead of a female volunteer with mixed skin (typized by a cosmetician and confirmed by sebometer measurements).

A very high percentage of Propionibacteria was determined by counting the fluorescence signals and comparing the result with the total cell count (>90%). A low percentage of Staphylococci was found (<10%). No Corynebacteria were found.

A microorganism sample was taken from the skin of another female volunteer by the sampling method described above.

The 16 S rRNA gene of a microorganism was isolated from one part of the sample. Subsequent sequence determination showed that the sequence was a new sequence although the microorganism could be assigned to the genus Corynebacterium. This sequence, on the basis of which a corresponding probe (according to SEQ ID NO. 21) that can detect this microorganism was developed, is shown under SEQ ID NO. 31 in the sequence protocol.

Another part of the sample was hybridized with the previously described bacteria-specific probe EUB and with a probe mixture (SEQ ID No. 07 to 11) for detection of the skin-relevant Corynebacteria.

A high percentage of Corynebacteria was determined by counting the fluorescence signals and comparing the result with the total cell count determined by the bacteria-specific probe (ca. 73%).

A small percentage (ca. 5%) of the Corynebacteria of this sample hybridized with the marked oligonucleotide according to SEQ ID NO. 21 which was determined by counting the fluorescence signals and comparing the result with the previously detected Corynebacteria count, the oligonucleotide according to SEQ ID NO. 22 being simultaneously used unmarked as competitor.

The disclosures of each patent, patent application, and publication cited or described in this document are hereby incorporated herein by reference, in their entireties.

Various modifications of the invention, in addition to those described herein, will be apparent to those skilled in the art from the foregoing description. Such modifications are also intended to fall within the scope of the appended claims.

1. An oligonucleotide having a sequence at least 80% homologous to at least one of SEQ. ID NO. 1 to 30, including sequences extended or deleted by one or more nucleotides, or sequences complementary thereto under stringent hybridization conditions.

2. A probe comprising the oligonucleotide of claim 1 and a detectable marker.

3. The probe of claim 2, wherein the detectable marker is covalently bonded to the oligonucleotide.

4. The probe of claim 2, wherein the detectable marker is a fluorescence marker, a chemoluminescence marker, a radioactive marker, an enzymatically active group, a hapten, or a nucleic acid detectable by hybridization.

5. The probe of claim 4, wherein the enzymatically active group is peroxidase or phosphatase.

6. The probe of claim 4, wherein the enzymatically active group is horse radish peroxidase or alkaline phosphatase.

7. The probe of claim 2, wherein the probe is specific for the genera Staphylococcus, Peptostreptococcus, Propionibacterium, Corynebacterium, Veillonella, Malassezia, or the Sporomusa taxon.

8. The probe of claim 2, further comprising a second oligonucleotide with a sequence other than that of the first oligonucleotide.

9. The probe of claim 8, wherein the second oligonucleotide has a sequence which provides for specific detection of bacteria of the genus Staphylococcus, Peptostreptococcus, Corynebacterium, Veillonella, Propionibacterium acnes, Malassezia, or the Sporomusa taxon.

10. The probe of claim 8, wherein the second oligonucleotide has a sequence at least 80% homologous to at least one of SEQ ID NO. 01, 02, 04, 07, 08, 10, 11, 13, 14, 16, 18 or 19 to 30.

11. The probe of claim 2, further comprising a plurality of oligonucleotides with sequences that are the same or different, and are at least 80% homologous to at least one of SEQ ID NO. 1 to 30.

12. A process for detecting microorganisms in a sample, comprising:
incubating the microorganisms with at least one probe according to claim 2; and
determining the presence of microorganisms hybridized with the probe.

13. The process of claim 12, further comprising quantifying the microorganisms.

14. The process of claim 12, wherein the microorganisms are of the genera Staphylococcus, Peptostreptococcus, Propionibacterium, Corynebacterium, Veillonella, Malassezia or the Sporomusa taxon.

15. The process of claim 12, wherein the sample is from a skin surface, a food, water, soil, air, wastewaters, a biofilm, a clinical examination material, a pharmaceutical, or a cosmetic product.

16. The process of claim 12, further comprising fixing the sample with denaturing reagents, crosslinking reagents, or heat.

17. The process of claim 12, further comprising immobilizing the microorganisms on a carrier.

18. The process of claim 12, further comprising permeabilizing the microorganisms.

19. The process of claim 18, wherein permeabilizing is carried out by partial degradation using cell-wall-lytic enzymes.

20. The process of claim 12, further comprising adding unmarked oligonucleotides.
